(19) United States
(12) Patent Application Publication
(10) Pub. No.: US 2024/0345029 A1
(43) Pub. Date: Oct. 17, 2024

(54) System and Method for Acoustic Measurement of Carbon Dioxide Concentration

(71) Applicant: John A. Cadwell, Richland, WA (US)

(72) Inventor: John A. Cadwell, Richland, WA (US)

(21) Appl. No.: 18/613,985

(22) Filed: Mar. 22, 2024

Related U.S. Application Data
(60) Provisional application No. 63/492,141, filed on Mar. 24, 2023.

(51) Int. Cl. G01N 29/024; A61B 5/08; G01N 29/22; G01N 29/32

(52) U.S. Cl. CPC G01N 29/024; A61B 5/0816; A61B 5/082; G01N 29/222; G01N 29/326; A61B 2562/0271; A61B 2562/029; G01N 2291/011; G01N 2291/021; G01N 2291/02809

(57) ABSTRACT

A system for measuring the carbon dioxide (CO2) concentration in an air sample. The system functions by buffering the air sample, measuring a transit time of sound in the air sample across a predefined distance, and determining a CO2 concentration in the air sample based on at least the temperature of the air sample and the determined transit time of the sound.

### CROSS-REFERENCE

The present specification relies on U.S. Provisional Patent Application No. 63/492,141, titled “System and Method for Acoustic Measurement of Carbon Dioxide Concentration”, filed on Mar. 24, 2023, for priority, the entirety of which is herein incorporated by reference in its entirety.

### FIELD

The present specification relates generally to a system and method for measuring a concentration of carbon dioxide (CO2) in a mixture. More particularly, the present specification relates to the use of the speed of sound travelling within a mixture containing CO2 for real-time measurement of fluctuating CO2 levels within the mixture.

### BACKGROUND

In various industrial and commercial applications, there is a need to measure carbon dioxide (CO2) concentrations in a volume of air. Healthcare applications such as, but not limited to, sleep studies, cardiac and other resuscitation situations, and procedures such as endotracheal intubation, require monitoring a patient's respiration and determining corresponding CO2 changes.

CO2 concentration measurement poses several challenges as the CO2 concentration in a sample of air/gases may fluctuate or change over time and may constitute as low as 1% of the sample air, in which case, it may be too difficult to measure with conventional technologies. A sample of human respiration comprises ambient, or air that is inhaled, and exhaled air. Such a sample may alternate between inhaled air and exhaled air at a rate of approximately 20 times per minute, in which case the CO2 levels in the sample may vary from a low of 1% to well over 10%.

Some commonly used techniques for measuring CO2 concentration changes at such rates include determining the optical absorption of CO2 in the far infrared range of light. In addition, systems using the attenuation of a transmitted infrared light source and systems using optical expansion due to heating by absorption of infrared energy are used commercially for measuring changing CO2 concentrations in a sample. One drawback is that both systems require calibrated infrared light sources. Further to that, infrared detectors may also be required. These conventional infrared light-based systems typically consume a large amount of power, are expensive, and are often prone to errors as optical absorption by other gases including water vapor and organic compounds (e.g. anesthetic gases) may provide erroneous results.

Still further, known systems for CO2 concentration measurement often require a vapor trap and mechanism for compensating for the effects of atmospheric pressure, increasing complexity, and cost of such systems. Further, compensation for atmospheric pressure fails above altitudes of 5000 feet rendering these systems ineffective at elevations higher than that. Usually, with optical infrared-based measuring systems, CO2 concentration sample measurements are taken at a rate of approximately 3 times per second in order to reduce the cost of infrared emitters employed in the systems, however higher rates are possible at a higher cost.

There is thus a need for a system and method for measuring CO2 concentration in air mixtures, that is efficient, cost effective, and can determine rapid changes in CO2 levels. What is also needed is a system and method that is simple to implement and can measure, in real-time, fluctuating CO2 concentrations within a sample.

### SUMMARY

The following embodiments and aspects thereof are described and illustrated in conjunction with systems, tools and methods, which are meant to be exemplary and illustrative, and not limiting in scope. The present application discloses numerous embodiments.

In some embodiments, the present specification describes a system for acoustic measurement of carbon dioxide (CO2) concentration in an air sample, the system comprising: a sampling tube configured to receive the air sample; a buffer chamber coupled with the sampling tube and configured to receive the air sample, wherein the buffer chamber includes a thermal and humidity buffer; a sensing chamber having a proximal end and a distal end and configured to receive the sample air, wherein the proximal end of the sensing chamber is coupled with the buffer chamber, wherein the sensing chamber comprises an acoustic transmitter positioned at the proximal end for transmitting sound waves that pass through the air sample in the sensing chamber, wherein the sensing chamber further comprises an acoustic reference receiver and an acoustic sample receiver coupled with at least one analog to digital converter (ADC) positioned at the distal end and configured to receive the transmitted sound waves and determine a transit time of the transmitted sound waves through the air sample; and a control unit configured to determine a CO2 concentration corresponding to the air sample based on at least the determined transit time of the sound waves through the air sample.

Optionally, the thermal and humidity buffer comprises a fine mesh substrate. Optionally, the fine mesh substrate has a mass greater than a thermal mass of the sample air.

Optionally, the system further comprises a temperature sensor coupled with the buffer chamber and configured to measure a temperature of the sample air after passing through the substrate.

Optionally, the system further comprises a humidity sensor coupled with the buffer chamber and configured to measure a humidity of the sample air after passing through the substrate.

Optionally, the system further comprises a barometric pressure sensor coupled with the buffer chamber and configured to measure a barometric pressure of the sample air after passing through the substrate. Optionally, the measured barometric pressure is used to convert a determined percentage of CO2 in the sample air to a partial pressure of CO2.

Optionally, the control unit is further configured to determine the CO2 concentration based on a measured temperature, humidity and barometric pressure of the sample air. Optionally, the control unit is further configured to determine a respiration rate using the air sample and based on at least the determined transit time of the sound waves through the air sample. Optionally, the system further comprises a vacuum pump coupled with the sensing chamber for removing the sample air from the system after determining the transit time of the sound. Optionally, the acoustic transmitter is an ultrasound transmitter and wherein the receiver is an ultrasound receiver.

Optionally, the sample air has a minimum residence time of 20 msec in the buffer chamber.

Optionally, the sensing chamber has a length ranging from 5 cm to 10 cm and is configured to contain a plurality of the transmitted sound waves.

Optionally, the sensing chamber has a cross sectional area ranging from 0.5 mm to 2.5 mm.

Optionally, the control unit is configured to execute a compensation algorithm that corrects for temperature and flow rate changes in the determination of the CO2 concentration.

Optionally, the acoustic transmitter is an ultrasonic acoustic pulse generator driven by a 40 kHz square wave driver configured to emit wavefronts, wherein the emitted wavefronts travel through the sensing chamber.

Optionally, the control unit is configured to execute an acoustic CO2 sensing algorithm, wherein the sensing algorithm uses at least one of the following inputs: transmitter control timing, arrival time of sound wave signal, pulse transit time, average aliasing time detection, concurrent temperature measurements, temperature compensation, pulse/signal change detection, signal level to CO2 level compensation, peak and valley detection, and respiratory rate calculation. Optionally, the acoustic CO2 sensing algorithm is configured to process input parameters to generate, as an output, at least a capnograph, a peak CO2 concentration, a minimum CO2 concentration, a respiratory rate and a temperature corresponding to the sample air.

In some embodiments, the present specification describes a method for acoustic measurement of carbon dioxide (CO2) concentration in a sample of air, the method comprising: maintaining a sample of air in a buffer chamber for a minimum residence time of 20 msec; transmitting a plurality of acoustic waves through the sample of air; measuring a transit time of the plurality of acoustic waves through the sample of air sample over a predefined distance; and determining a CO2 concentration in the sample of air based on at least a temperature of the sample of air and the measured transit time of the plurality of acoustic waves.

Optionally, the temperature of the sample of air is measured by a temperature sensor.

Optionally, the method further comprises receiving the sample of air into a sample tube; passing the sample of air into the buffer chamber, wherein the buffer chamber comprises a fine mesh substrate; and passing the sample of air into a sampling chamber where the plurality of acoustic waves are transmitted through the sample of air.

Optionally, the fine mesh substrate is heated to a predefined temperature.

Optionally, the temperature of the sample of air is equal to the temperature of the fine mesh substrate.

Optionally, the method further comprises passing the sample of air over a temperature sensor upon exiting the buffer chamber to measure the temperature of the sample of air.

Optionally, the method further comprises passing the sample of air, upon exiting the buffer chamber, over a humidity sensor to measure humidity of the sample of air and a barometric pressure sensor to measure barometric pressure of the sample of air.

Optionally, transmitting the plurality of acoustic waves through the sample of air comprises: causing an acoustic pulse to travel through the sample of air in the sampling chamber; and receiving the acoustic pulse at a receiver.

Optionally, the method further comprises removing the sample of air out of the sampling chamber using a vacuum pump.

Optionally, the method further comprises using a muffler to reduce the vacuum pump noise artifact from the acoustic waves.

Optionally, determining the CO2 concentration in the sample of air comprises executing a compensation algorithm that corrects for temperature and flow rate changes.

Optionally, the method further comprises performing a calibration using atmospheric air to represent a known amount of CO2 concentration.

Optionally, determining the CO2 concentration in the sample of air comprises executing an acoustic CO2 sensing algorithm, using the control unit, wherein the sensing algorithm uses at least one of the following inputs: transmitter control timing, arrival time of sound wave signal, pulse transit time, average aliasing time detection, concurrent temperature measurements, temperature compensation, pulse/signal change detection, signal level to CO2 level compensation, peak and valley detection, and respiratory rate calculation, and generating as an output at least a capnograph, a peak CO2 concentration, a minimum CO2 concentration, a respiratory rate and a temperature corresponding to the sample of air.

The aforementioned and other embodiments of the present specification shall be described in greater depth in the drawings and detailed description provided below.

### BRIEF DESCRIPTION OF THE DRAWINGS

The accompanying drawings illustrate various embodiments of systems, methods, and various other aspects of the disclosure. Any person with ordinary skills in the art will appreciate that the illustrated element boundaries (e.g. boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1A is a block diagram illustrating a system for the acoustic measurement of carbon dioxide, in accordance with an embodiment of the present specification;

FIG. 1B is a block diagram illustrating a system configuration for the acoustic measurement of carbon dioxide, in accordance with an embodiment of the present specification;

FIG. 1C illustrates a nasal cannula that may be used to connect the system for the acoustic measurement of carbon dioxide described with respect to FIG. 1A, to a user/patient, in accordance with an embodiment of the present specification;

FIG. 2 is a flowchart detailing steps for performing the acoustic measurement of carbon dioxide, in accordance with an embodiment of the present specification;

FIG. 3A illustrates a pump-less down draft sensing system configuration for the acoustic measurement of carbon dioxide, in accordance with an embodiment of the present specification; and

FIG. 3B illustrates a pump-less side draft sensing system configuration for the acoustic measurement of carbon dioxide, in accordance with an embodiment of the present specification.

### DETAILED DESCRIPTION

In various embodiments, the present specification describes systems and methods for measuring a concentration of carbon dioxide (CO2) in a sample by measuring a transit time of a sound wave travelling over a predefined distance within the air sample. Velocity is determined by dividing the transit time by the predefined distance, whereby the determined velocity is then used to determine the CO2 concentration in the air sample. In embodiments, the CO2 concentration is determined by using i) the determined velocity and ii) the ratio of the speed of sound in CO2 to the speed of sound in air. The speed of sound in an air sample mixture is proportional to the ratio of speed of sound in CO2 to the speed of sound in air wherein the speed of sound in CO2 is approximately 30% slower than the speed of sound in air.

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

In the description and claims of the application, each of the words “comprise”, “include”, “have”, “contain”, and forms thereof, are not necessarily limited to members in a list with which the words may be associated. Thus, they are intended to be equivalent in meaning and be open-ended in that an item or items following any one of these words is not meant to be an exhaustive listing of such item or items, or meant to be limited to only the listed item or items. It should be noted herein that any feature or component described in association with a specific embodiment may be used and implemented with any other embodiment unless clearly indicated otherwise.

It must also be noted that as used herein and in the appended claims, the singular forms “a,” “an,” and “the” include plural references unless the context dictates otherwise. Although any systems and methods similar or equivalent to those described herein can be used in the practice or testing of embodiments of the present disclosure, the preferred, systems and methods are now described.

In embodiments, the present specification describes a system and method for measuring, in real-time, rapidly changing carbon dioxide (CO2) levels in a sample, by using the ratio of the speed of sound in air to the speed of sound in CO2 as measured across a known distance to compute the fraction of CO2 contained in the sample.

In embodiments, the system may optionally be calibrated to verify the accuracy of CO2 measurement. It should be noted that the amount of atmospheric CO2 is negligible. Further, the speed of sound is a physical constant of the gases, therefore it can be assumed to not materially change. Thus, air may be used to verify the near zero or negligible CO2 concentration measured by the system prior to testing an actual sample for CO2. In some embodiments, the minimum CO2 concentration may be assumed to be zero. A measurement of actual zero is preferably used to verify operation of the system.

A source of potential error in the system is the jitter of the pulse timing measurement system. However, jitter in the timing measurement is nominal and is not affected by an amplitude of the driving signal. As such, the jitter need not be specifically calibrated for. This feature is very different from optical methods where the light intensity needs to be carefully controlled. In one embodiment, the jitter of a timing measurement circuit may be compensated for by sampling at extremely high rates and averaging the responses.

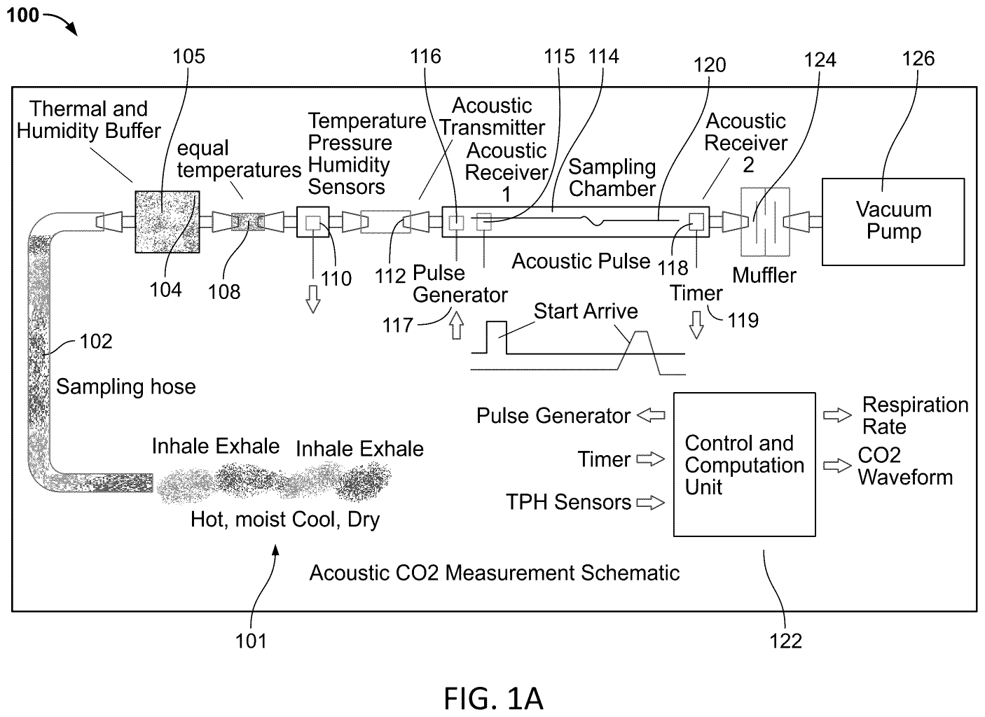

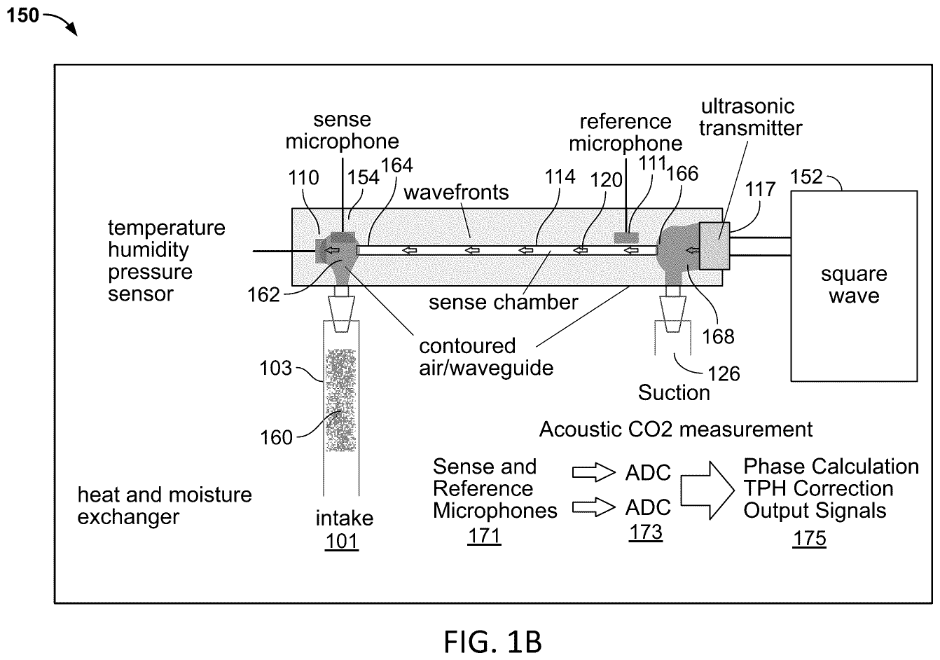

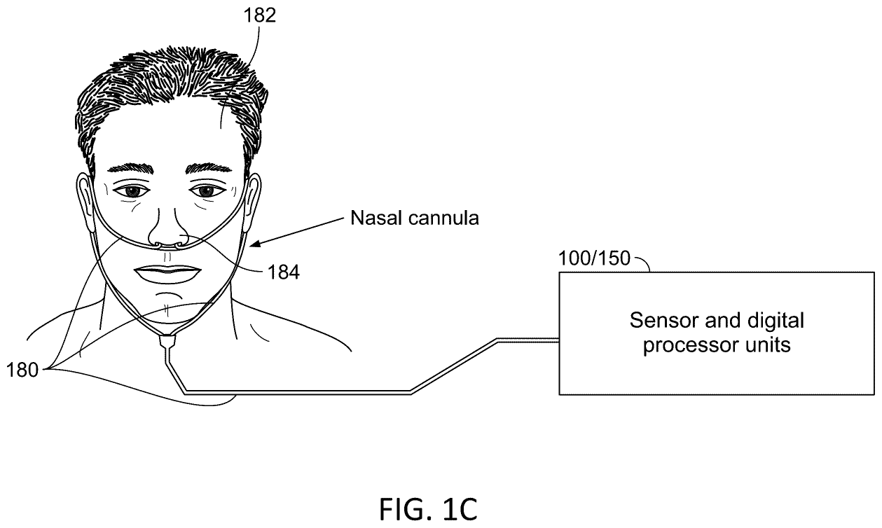

FIG. 1A is a block diagram illustrating a system for acoustic measurement of carbon dioxide concentration (determining the fraction of CO2 in an air mixture sample), in accordance with an embodiment of the present specification. System 100 comprises a sampling hose 102, which, in an embodiment is a tube into which a subject inhales and exhales, thereby moving sample air 101 through the hose 102. In embodiments, sample air 101 is hot and moist during an exhale and cool and dry during an inhale. In an embodiment, system 100 is a continuous sampling system.

Sampling hose 102 is used to transport the sample air 101 to a buffer chamber 104, which is a thermal and humidity buffer and comprises a moist fine mesh substrate 105. In embodiments, changes in the temperature and humidity of an air sample are reduced by using thermal and humidity buffering obtained through buffer chamber 104 and via the fine mesh substrate 105, which, in an embodiment is a buffering moist mesh substrate material, whereupon the resulting temperature and humidity are determined and compensated for. In embodiments, the buffering moist mesh substrate material 105 may comprise materials such as, but not limited to acrylic, polyester, rayon, cotton or wire wool.

In embodiments, an optimal mass and configuration of the fine mesh substrate 105 is based on the moisture content in the sample air 101, as moisture or water vapor is the largest carrier of heat, and the specific heat of water vapor is approximately twice that of air. Aspects of the present specification employ acoustics for the measurement of CO2 concentration, and, as is known by persons of ordinary skill in the art that temperature and humidity changes affect the speed of sound waves traveling through air, especially at higher temperatures and humidity. For example, the speed of sound travelling through air changes by approximately 0.17% per degree centigrade change in air temperature. Further, humidity (or the change of the percentage of water vapor in the air/gas mixture) affects the speed of sound because the speed of sound in water vapor is more than in air. During normal breathing, the temperature of inhaled air at ambient temperature is approximately 20 degrees centigrade, while moist exhaled air/breath has a temperature of approximately 37 degrees centigrade.

At least a fraction of the sample air 101 comprises carbon dioxide (CO2). In embodiments, a mass of the fine mesh substrate 105 is greater than the thermal mass of the sample air 101. The sample air 101 comprising at least a fraction of CO2 passes relatively unimpeded through the fine mesh substrate 105, and during passage, a temperature of the sample air 101 becomes equal to the temperature of the fine mesh substrate 105, in a portion 108 of the sample tube, due to the large surface area of the substrate 105. In an embodiment, the sample air 101 passes over one or more sensors 110 such as, but not limited to a temperature sensor, a humidity sensor, and a barometric pressure sensor, which measure the temperature, humidity, and pressure of the sample air 101, respectively, upon exiting the fine mesh substrate 105.

In embodiments, barometric pressure may be used to determine the percentage of CO2 in the air sample for any given partial pressure of CO2. Barometric pressure is nominally constant from breath-to-breath and is determined primarily by altitude above sea level.

In embodiments, a time constant of the temperature sensor 110 is much smaller than the rate of temperature change of the temperature buffering fine mesh substrate 105. In an embodiment, the time constant of the buffering fine mesh substrate 105 is 60 seconds and the time constant of the temperature sensor ranges between 2-5 seconds.

In an embodiment, a temperature of the fine mesh substrate 105 is used to determine the temperature of the sample air 101 for subsequent calculations. Optionally, a temperature correction that is based on the actual measured sample air temperature may be applied to subsequent calculations. In embodiments, the temperature of the incoming sample air 101 converges towards the average temperature of the fine mesh substrate 105. Since a mass of the fine mesh substrate 105 is large, the rate of change of temperature is much slower than the response time of a temperature measuring system. Thus, the temperature of the sample air 101 exiting the fine mesh substrate 105, whether heated or cooled during transit approximates the average temperature resulting in negligible errors in temperature measurement. In embodiments of the present specification, buffering the temperature of the sample air by using the fine mesh substrate 105 allows the temperature sensor 110 to have a response time slower than would be required if the sample air 101 was not buffered. Stated differently, because the sample air can be assumed to converge toward the relatively stable and known thermal buffer temperature, the temperature sensor response time becomes less critical. In some embodiments a temperature sensor having a faster response may be used for determining the temperature of the sample air 101. In an embodiment the thermal buffer has a time constant of 30 seconds and the temperature sensor a time constant of 5 seconds.

After temperature measurement the sample air 101 passes to a sensing/sampling chamber 114 via a portion 112 of the sample tubing. The sampling chamber 114 comprises an acoustic transmitter 116 positioned at a proximal end of the sampling chamber 114, a first reference acoustic receiver 115 and a second acoustic receiver 118 positioned at a predefined distance at a distal end of sampling chamber 114 as shown in FIG. 1A. The acoustic transmitter 116 and acoustic receiver 118 are enclosed in the sampling chamber 114, wherein the channel acts as both an acoustic waveguide and a fluid flow path. As the sample air 101 passes through the sampling chamber 114, an acoustic pulse 120 generated by a pulse generator 117 coupled with the acoustic transmitter 116 is transmitted by the acoustic transmitter 116. The pulse 120 travels through the channel, is received by acoustic receivers 115 and 118 and is sampled by an analog to digital converter (ADC) 119 to detect and measure a transit time of the pulse 120. In embodiments, the generation and detection of pulse 120 is performed repeatedly, to obtain a predefined number of sequential measurements. In an embodiment, approximately 100 samples per data point are collected which improves the signal to noise ratio of the system by a factor of approximately 10 and provides an output of 400 samples/second. Accordingly, in an embodiment, the system outputs approximately 10-50 readings per second.

The changes in transit times of the pulse 120 through the sample air 101 are reflective of the instantaneous CO2 concentration of the sample air 101. As the CO2 levels in the sample air 101 change, the sampling chamber 114 comprises a mixture of samples. Therefore, in embodiments, sequential minimum and maximum measured transit times are used to obtain final CO2 levels in the sample air 101. In an embodiment a weighted numerical mean averaging is implemented whereby a generic formula or calculation can be represented by:

Mean=k1*samples0 . . . n+k2*samples n+1 . . . 2n+k3*samples 2n+1 . . . 3n+ . . . /scaleFactor [Equation 1]

Where, k1>k2>k3>(k4) and scaleFactor is k1*n+k2*n+k3*n+(k4*n).

In an embodiment, a control and computation unit 122 is configured to execute a compensation algorithm to correct for the effects of temperature, humidity and barometric pressure, and determine the changing CO2 levels in the sample air 101. In an embodiment, the compensation algorithm is a linear extrapolation algorithm.

The control and computation unit 122 is configured to input parameters obtained from the pulse generator 117, the ADC 119, and the temperature sensor 110, and to execute a predefined compensation algorithm based on the inputs. The control and computation unit 122, based on the inputs, is configured to output a determined CO2 waveform and a respiration rate of the subject corresponding to the air sample 101. In an embodiment, sequential digital CO2 values are converted to a changing voltage (for example, ranging from 0 to 1 volts), which represents the respiratory CO2. The peak values and the minimum values in the sequence occur once per breath, and the time between two consecutive peaks or between two consecutive valleys provides the respiration rate. In an embodiment, an air flow rate of the sample air 101 is also input to the control and computation unit 122 for obtaining additional compensation to improve the accuracy of the determined respiration rate and CO2 waveform.

In an embodiment, the following computational algorithm may be used for temperature, humidity, and pressure correction, computation of a CO2 fraction, and conversion to partial pressure (temperature calculations and velocities are normalized to 0 degrees C.) wherein,
V is velocity or speed of sound;
k1 is the rate of change of velocity per degree C.;
k2 is the rate of change of velocity per fraction of water vapor;
k3 is the conversion constant from fractionCO2 to partialPressureCO2;
fractionH2O is obtained from the published mass of H2O at temperature, humidity, and pressure [fractionH2O=fx(humidity, temperature, barometricPressure)];
VSoundAir and VsoundCO2 are the known values of the speed of sound in air and CO2;
PartialPressureCO2(measuredSpeedOfSound, temperature, humidity, barometricPressure);

$$VMixture = measured Speed ofSound * ( 1 + k 1 * temperature ) \qquad [ Equation 2 ]$$

$$VAirPlus CO 2 = VMixture * ( 1 + k 2 * fraction H 2 O ) \qquad [ Equation 3 ]$$

$$VAirPlus CO 2 = fraction CO2 * VSound CO2 + fractionAir * VSoundAir \qquad [ Equation 4 ]$$

$$fractionAir = 1 - fraction CO 2 \qquad [ Equation 5 ]$$

$$fraction CO2 = ( VAirPlus CO2 - VSoundAir ) / ( VSound CO2 - VSoundAir ) ; \qquad [ Equation 6 ]$$

$$PartialPressure CO 2 = k 3 * fraction CO2 * barometricPressure \qquad [ Equation 7 ]$$

In an embodiment, the sampling chamber 114 is coupled with one or more means for obstruction detection, to detect obstruction such as but not limited to kinked, crimped, or flow-impeded hoses, which is a known possible error condition. In an embodiment, a barometric pressure sensor 110 reading shows a drop in pressure of the sample air 101 in the presence of obstruction. The barometric pressure sensor 110, when proximal to a kink in a hose, shows a dramatic drop in pressure due to obstruction. In another embodiment, the present specification allows for the measurement of vacuum pump current. When obstructed, the pump itself would change and the change would indicate inadequate air flow.

In embodiments, air samples may be corrected for errors in flow by using time-stamped sample data, air flow measurement data, and tube geometry data. In embodiments, up to a 20% error in air flow measurement does not adversely affect the acoustic measurement of CO2. After measurement the sample air 101 is ejected out of, or removed from, the system 100 via a vacuum pump 126. In an embodiment, a muffler 124 is placed in-line with the vacuum pump to reduce noise made by the vacuum pump 126.

In embodiments, the control and computation unit 122 is configured to execute an acoustic CO2 sensing algorithm. Parameters such as, but not limited to: transmitter control timing, arrival time of pulse/wave signal, pulse transit time, average aliasing time detection, concurrent temperature measurements, temperature compensation, pulse/signal change detection, signal level to CO2 level compensation, peak and valley detection, and respiratory rate calculation are input to the acoustic CO2 sensing algorithm, which is configured to process the input parameters to output at least a capnograph, a peak CO2 concentration, minimum CO2 concentration, a respiratory rate and a temperature corresponding to the sample air.

In an embodiment, some of the above parameters may be determined and used as follows:
‘Average’=sum of n measurements/n. The parameter ‘average’ reduces noise by sqrt(n) and reduces sample rate by n.
‘Aliasing time detection’: phase is inverseTan(signal) which aliases (wraps) every m*Pi, causing an abrupt and erroneous change in value, which may be avoided by limiting allowable gas mixtures, and detected by abrupt non-physiological changes.
‘Concurrent temperature measurements’: temperature, humidity and pressure are measured at the same time as the velocity and are used to compute the partial pressure as described above.
‘Pulse/signal change detection’: failure to breathe for an extended period of time is revealed in the loss of periodic rising/falling signal and may be determined and reported by the sensing algorithm.
‘Peak and valley detection’: the sensing algorithm monitors sequential maximum CO2 and minimum CO2 levels.
‘Respiratory rate’: 1/(time interval between two consecutive peaks in a CO2 waveform) and may be plotted over time intervals, such as, but not limited to, every second for a 1 minute or 5 minutes time interval.

In an embodiment, a size of the sampling chamber 114, including a length and cross-sectional dimension affects the flow rate of sample air 101. A smaller than optimal cross-section of the sampling chamber 114 would reduce transit time of the sample air 101 but would result in an increased acoustic attenuation, which may provide inaccurate flow rate measurement. On the other hand, a longer than optimal sampling chamber 114 may provide an improved signal to noise ratio, but may reduce sampling frequency. In embodiments, a length of the sampling chamber 114 ranges from 5 cm to 10 cm and a cross-sectional diameter of the sampling chamber 114 ranges from 1.5 mm to 4 mm. In an embodiment, a sampling chamber having a diameter of 1.5 mm results in a 60% attenuation as compared to a sampling chamber having a diameter of 4 mm. In embodiments, the sampling chamber 114 is designed to promote laminar flow, as turbulence in the sample air 101 flow generates acoustic noise which degrades signal to noise ratio and the quality of the measurement. Therefore, the sampling chamber 114 is designed to be smooth-walled having a geometry comprising smooth transitions for enabling flow, wherein the flow rate is controlled to avoid turbulence. In embodiments, the sampling chamber 114 may be made from materials such as, but not limited to, copper, brass, steel, and a variety of plastics.

FIG. 1B is a block diagram illustrating a specific system configuration for the acoustic measurement of carbon dioxide as described with respect to FIG. 1A and provided for exemplary purposes, in accordance with an embodiment of the present specification. System 150 comprises acoustic pulse generator 117, which, in an embodiment, is a 40 kHz highly resonant ultrasonic transmitter driven by a 40 kHz square wave driver 152. In different embodiments, square wave drivers at any frequency above 20 kHz may be used. The acoustic pulse generator 117 is configured to emit pulses/wavefronts 120 which travel through a sampling chamber 114, which in an embodiment is a metal tube having a length designed to contain a plurality of the emitted wavefronts simultaneously. In an embodiment, the sampling chamber 114 is 5 wavelengths long wherein the length of one wavelength is 7 mm at 40 kHz frequency. The use of a longer tube yields a greater change in latency than a shorter tube, and the use of multiple concurrent wavefronts allows measuring the latency shift 40,000 times per second so that the measurements can be subsequently averaged to improve SNR. In an embodiment, the sampling chamber 114 is a metal tubing having an internal diameter of 2 mm and a length of 8 cm. The dimensions of the sampling chamber 114 provide a tradeoff between acoustic attenuation and air flow transit time through the tube. In an embodiment, the sampling chamber 114 is flushed within 50 msec.

In an embodiment the pulse/wavefronts 120 are sinusoidal and can be detected by a high frequency response microphone 154 having a 40 kHz frequency response, which functions as the receiver 118 (shown in FIG. 1A). In an embodiment, the resonance of the transmitter 116 ranges from 39 KHz to 41 KHz. Since, the phase of the wavefronts 120 relative to the driving signal depends on the actual resonant frequency of each specific transmitter, in order to eliminate transmitter phase error, in an embodiment, a reference microphone 111 is placed in close proximity to the transmitter 116 for measuring a ‘starting’ phase of the acoustic signal. The acoustic generator 117 and the microphone 154 are positioned on opposite ends of the sampling chamber 114, as shown in FIG. 1B. An output 171 of the microphone 154 is filtered by a filter in order to reduce low frequency noise and subsequently digitally sampled by an ADC 173 to determine the transit time of the wavefront 120 and obtain an output signal 175. Due to system design, the sampled wavefront 120 being sensed by the ADC 173 was generated by the acoustic generator 117 at an earlier time, which may be a plurality of samples earlier. The maximum tube length is limited to prevent aliasing which happens when a full wavelength change in latency occurs at maximum measurable CO2 levels. In an embodiment, the sampling chamber 114 is less than 2.5 wavelengths long, which translates to the sampling tube having a length of approximately 2 inches at 40 kHz wavefront frequency, as this length prevents aliasing if the CO2 level in the air sample is 100%. In another embodiment the tube length is increased to improve accuracy, which requires detecting and correcting for aliasing to measure CO2 values up to 100%. In this embodiment, a response of a zero-crossing detector (such as detector 158 shown in FIG. 1A) is aliased by several wavelengths, which is taken into account while processing and calculating CO2 transit time. In an embodiment, the starting of wavefronts and arrival of samples is aliased and measured approximately 5 full wavefronts apart; which difference in time calculation is taken into account by the processing algorithm.

In the embodiment shown in FIG. 1B, sample air 101 comprising at least a fraction of CO2 is pushed in an inlet tubing 103 that includes a porous wad of moist material 160 for thermal absorption and buffering (as explained with reference to FIG. 1A above), wherein a distal end of the inlet tubing 103 is coupled with a first end of a first contoured wave guide 162. A second end of the first contoured wave guide 162 is coupled to a proximal end 164 of the sampling chamber 114. The waveguides expand and contract smoothly to avoid abrupt acoustic impedance changes. Abrupt changes will cause attenuation and reflection which degrade the signal as is known to those skilled in the art. The second end of the first contoured wave guide 162 is also coupled with sensors 110 and the microphone 154. A distal end 166 of the sampling chamber 114 is coupled with a first end of a second contoured wave guide 168 which, at a second end is coupled with the acoustic generator 117 comprising the transmitter and a vacuum pump 126, for flushing out the sample air 101 from the sampling chamber 114.

FIG. 1C illustrates a nasal cannula that may be used to connect the system for acoustic measurement of carbon dioxide described with respect to FIG. 1A, with a user/patient, in accordance with an embodiment of the present specification. As shown, a cannula 180 is coupled with a patient's 182 nose/nasal passage 184, such that the patient's 182 inhales and exhales ambient air, and the inhales and exhales are captured as sample air for input to the system 100/150 (as shown in FIG. 1A, 1B) for acoustic measurement of CO2.

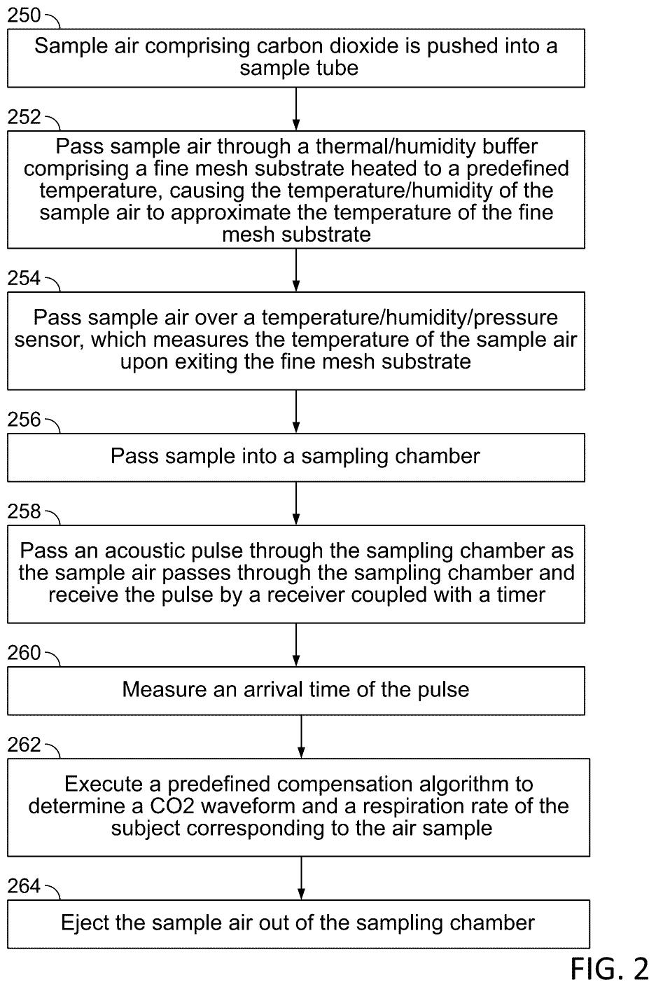

FIG. 2 is a flowchart illustrating the steps of operation for acoustic measurement of carbon dioxide, in accordance with an embodiment of the present specification. At step 250, sample air comprising carbon dioxide is pushed into a sample tube. In embodiments, sample air comprises hot and moist air obtained from a subject's exhale and cool and dry air obtained from the subject's inhale. At step 252 the sample air passes through a thermal buffer comprising a moist fine mesh substrate causing the temperature and humidity of the sample air to approximate the temperature and humidity of the fine mesh substrate. At step 254 the sample air passes over a temperature sensor, which measures the temperature of the sample air upon exiting the fine mesh substrate. In an embodiment, a temperature of the fine mesh substrate is measured for determining the temperature of the sample air, and temperature correction is applied to subsequent measurements. In embodiments, temperature of the incoming sample air converges toward the average temperature of the fine mesh substrate. Since, a mass of the fine mesh substrate is large, the rate of change of temperature is much slower than the response time of a temperature measuring system. In such a case, the temperature of the sample air exiting the fine mesh substrate, whether heated or cooled during transit is proximate the average temperature resulting in negligible errors in temperature measurement.

At step 256 the sample air passes into a sampling chamber. At step 258, as the sample air passes through the sampling chamber, an acoustic pulse is generated and travels through the sampling chamber and is subsequently received by a receiver coupled with an ADC. At step 260 the ADC samples and measures the arrival time of the pulse. In embodiments, the generation and detection of the acoustic pulse is performed repeatedly, to obtain a predefined number of sequential measurements. Changes in transit times of the pulse through the sample air reflect the instantaneous CO2 concentration of the sample air. As the CO2 levels in the sample air change, the sampling chamber comprises a mixture of samples. Therefore, in embodiments, sequential minimum and maximum measured arrival times are used to obtain final CO2 levels in the sample air.

At step 262 a predefined compensation algorithm is executed and applied to determine a CO2 waveform and a respiration rate of the subject corresponding to the air sample. In embodiments, parameters such as, but not limited to, pulse parameter data, ADC data, temperature, humidity and barometric pressure sensor data are input into the predefined compensation algorithm. At step 264 the sample air is pulled out of the sampling chamber via a vacuum pump.

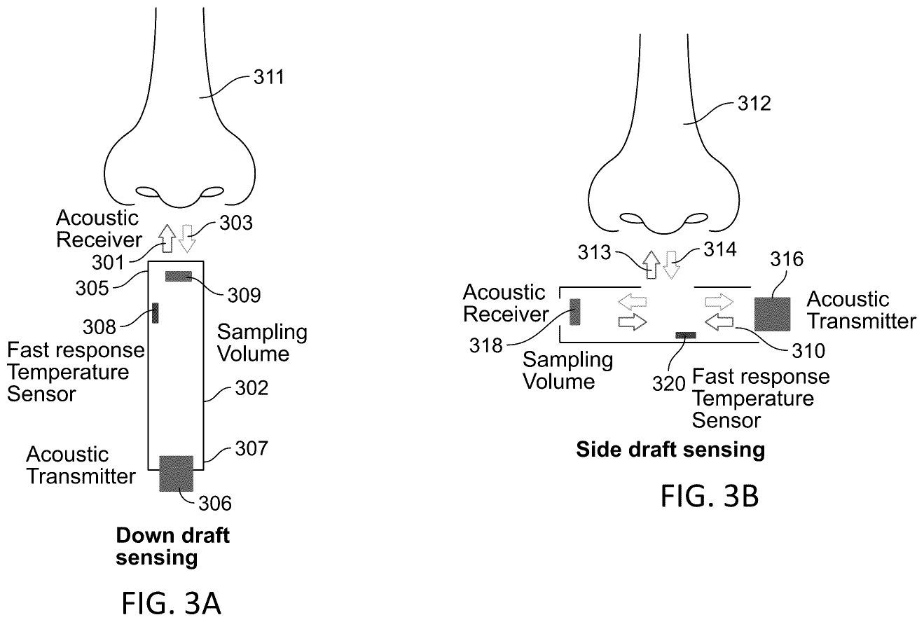

FIG. 3A illustrates a pump-less down draft sensing system configuration for acoustic measurement of carbon dioxide, in accordance with an embodiment of the present specification. A sampling tube is positioned below a subject's nostrils 311, parallel to a direction of inhale 301 and exhale 303. Sample air is inhaled 301 and exhaled 303 through the sampling tube 302 comprising an acoustic transmitter 306 positioned at a distal end 307 for transmitting sound waves through the sampling tube 302. The transmitted sound waves are received by an acoustic receiver 309 positioned at a proximal end 305 of the sampling tube 302. A fast response temperature sensor 308 is provided in the sampling tube 302 for measuring the temperature of the inhaled and exhaled sample air within the sampling tube 302. CO2 concentration in the sample air volume is determined based on the transit time of the sound waves through the sample volume as well as the temperature of the sample volume, as is described with reference to FIGS. 1A and 1B.

FIG. 3B illustrates a pump-less side draft sensing system configuration for acoustic measurement of carbon dioxide, in accordance with an embodiment of the present specification. As shown in FIG. 3B, a sampling tube 310 is positioned perpendicular to a subject's nostrils 312 and a direction of inhale 313 and exhale 314 by the subject. Sample air is inhaled and exhaled through an opening provided in the center of the tube 310 as shown in FIG. 3B. The sampling tube 310 comprising an acoustic transmitter 316 for transmitting sound waves through the sampling tube 302 and an acoustic receiver 318 for receiving the transmitted waves, wherein the transmitter 316 and the receiver 318 are positioned at opposing ends of the sampling tube 310. A fast response temperature sensor 320 is provided in the sampling tube 310 for measuring the temperature of the inhaled and exhaled sample air within the sampling tube 310. CO2 concentration in the sample air volume is determined based on the transit time of the sound waves through the sample volume as well as the temperature of the sample volume, as is described with reference to FIG. 1.

As is known to those skilled in the art, generation of acoustic pulses; measurement of phase shift and transit time; and measurement of temperature and flow rates may be performed in a plurality of ways, all of which are covered under the scope of the present specification. In various embodiments, different types of configurations of length, geometry, sample rates and transducers may be used to achieve CO2 concentration measurement.

The above examples are merely illustrative of the many applications of the system and method of the present specification. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

What is claimed is:

1. A system for acoustic measurement of carbon dioxide (CO2) concentration in an air sample, the system comprising:
a sampling tube configured to receive the air sample;
a buffer chamber coupled with the sampling tube and configured to receive the air sample, wherein the buffer chamber includes a thermal and humidity buffer;
a sensing chamber having a proximal end and a distal end and configured to receive the sample air, wherein the proximal end of the sensing chamber is coupled with the buffer chamber, wherein the sensing chamber comprises an acoustic transmitter positioned at the proximal end for transmitting sound waves that pass through the air sample in the sensing chamber, wherein the sensing chamber further comprises an acoustic reference receiver and an acoustic sample receiver coupled with at least one analog to digital converter (ADC) positioned at the distal end and configured to receive the transmitted sound waves and determine a transit time of the transmitted sound waves through the air sample; and
a control unit configured to determine a CO2 concentration corresponding to the air sample based on at least the determined transit time of the sound waves through the air sample.

2. The system of claim 1, wherein the thermal and humidity buffer comprises a fine mesh substrate.

3. The system of claim 2, wherein the fine mesh substrate has a mass greater than a thermal mass of the sample air.

4. The system of claim 2, further comprising a temperature sensor coupled with the buffer chamber and configured to measure a temperature of the sample air after passing through the substrate.

5. The system of claim 2, further comprising a humidity sensor coupled with the buffer chamber and configured to measure a humidity of the sample air after passing through the substrate.

6. The system of claim 2, further comprising a barometric pressure sensor coupled with the buffer chamber and configured to measure a barometric pressure of the sample air after passing through the substrate.

7. The system of claim 5, wherein the measured barometric pressure is used to convert a determined percentage of CO2 in the sample air to a partial pressure of CO2.

8. The system of claim 1, wherein the control unit is further configured to determine the CO2 concentration based on a measured temperature, humidity and barometric pressure of the sample air.

9. The system of claim 1, wherein the control unit is further configured to determine a respiration rate using the air sample and based on at least the determined transit time of the sound waves through the air sample.

10. The system of claim 1, further comprising a vacuum pump coupled with the sensing chamber for removing the sample air from the system after determining the transit time of the sound.

11. The system of claim 1, wherein the acoustic transmitter is an ultrasound transmitter and wherein the receiver is an ultrasound receiver.

12. The system of claim 1, wherein the sample air has a minimum residence time of 20 msec in the buffer chamber.

13. The system of claim 1, wherein the sensing chamber has a length ranging from 5 cm to 10 cm and is configured to contain a plurality of the transmitted sound waves.

14. The system of claim 1, wherein the sensing chamber has a cross sectional area ranging from 0.5 mm to 2.5 mm.

15. The system of claim 1, wherein the control unit is configured to execute a compensation algorithm that corrects for temperature and flow rate changes in the determination of the CO2 concentration.

16. The system of claim 1, wherein the acoustic transmitter is an ultrasonic acoustic pulse generator driven by a 40 kHz square wave driver configured to emit wavefronts, wherein the emitted wavefronts travel through the sensing chamber.

17. The system of claim 1, wherein the control unit is configured to execute an acoustic CO2 sensing algorithm, wherein the sensing algorithm uses at least one of the following inputs: transmitter control timing, arrival time of sound wave signal, pulse transit time, average aliasing time detection, concurrent temperature measurements, temperature compensation, pulse/signal change detection, signal level to CO2 level compensation, peak and valley detection, and respiratory rate calculation.

18. The system of claim 17, wherein the acoustic CO2 sensing algorithm is configured to process input parameters to generate, as an output, at least a capnograph, a peak CO2 concentration, a minimum CO2 concentration, a respiratory rate and a temperature corresponding to the sample air.
